(19) United States
(12) Patent Application Publication
(10) Pub. No.: US 2020/0297892 A1
(43) Pub. Date: Sep. 24, 2020

(54) ANTIMICROBIAL WOUND DRESSING

(71) Applicant: KCI USA, INC., San Antonio, TX (US)

(72) Inventor: Derek Walter SILCOCK, Skipton (GB)

(21) Appl. No.: 16/085,671

(22) PCT Filed: Mar. 15, 2017
(86) PCT No.: PCT/US2017/022577

§ 371 Date: Sep. 17, 2018

Related U.S. Application Data
(60) Provisional application No. 62/310,270, filed on Mar. 18, 2016.

(51) Int. Cl. A61L 15/28; A61L 15/32; A61L 15/58

(52) U.S. Cl. CPC A61L 15/28; A61L 15/325; A61L 2300/404; A61L 2300/104; A61L 15/58

(57) ABSTRACT

Wound dressing compositions comprising: carboxymethyl cellulose, oxidized regenerated cellulose (ORC), and silver. The dressings optionally further comprise non-gelling cellulose fibers. The compositions may comprise a complex of the ORC and silver. Wound dressings are also provided, comprising an absorbent layer comprising the wound dressing compositions.

### RELATED APPLICATIONS

The present invention claims the benefit, under 35 USC § 119(e), of the filing of U.S. Provisional Patent Application Ser. No. 62/310,270, entitled “Antimicrobial Wound Dressing,” filed Mar. 18, 2016. This provisional application is incorporated herein by reference for all purposes.

### TECHNICAL FIELD

The present technology relates to compositions and devices, including wound dressings, for application to wounds.

### BACKGROUND

A wide variety of materials and devices, generally characterized as “wound dressings,” are known in the art for use in treating an injury or other disruption of tissue. Such wounds may be the result of trauma, surgery, or disease, and affect skin or other tissues. In general, dressings may control bleeding, absorb wound exudate, ease pain, assist in debriding the wound, protect wound tissue from infection, or otherwise promote healing and protect the wound from further damage.

In particular, many wound dressings protect, or assist in the treatment of, infections associated with wounds. Infections can retard wound healing and, if untreated, can result in tissue loss, systemic infections, septic shock and death. A variety of dressings containing antimicrobial agents are known in the art. Nevertheless, there remains a need for improved dressings having one or more characteristics of improved antimicrobial efficacy, improved wound healing, improved absorption of blood and wound exudate, improved wound protection, reduced cost, and greater ease of use.

### BRIEF SUMMARY

The present technology provides wound dressing compositions comprising carboxy methyl cellulose, oxidized regenerated cellulose and silver. In various embodiments, the compositions may also contain non-gelling cellulose fibers.

The oxidized regenerated cellulose may be complexed with the silver, forming a complex having from about 10% to about 40% of silver in some embodiments.

The compositions may contain an optional hydrogel, such as a polyurethane gel, hydroxyethyl cellulose, hydroxylpropyl cellulose, hydroxypropylmethyl cellulose, modified acrylamide polymer, alginate, pectin, galactomannan, chitosan, gelatin, hyaluronate, or mixture thereof. In some embodiments, the composition further comprises collagen. Compositions may also contain optional wound healing active materials.

The present technology also provides wound dressings comprising a wound dressing composition. The wound dressing composition may be a component of an absorbent layer in the dressing. The absorbent layer may be in sheet form. The dressing may further comprise a backing sheet having an adhesive margin, and may have an apertured top sheet.

### DRAWING

FIG. 1 is a perspective view of a wound dressing according to the present technology.

It should be noted that the FIGURE set forth herein is intended to exemplify the general characteristics of materials and methods among those of the present technology, for the purpose of the description of certain embodiments. The FIGURE may not precisely reflect the characteristics of any given embodiment, and are not necessarily intended to define or limit specific embodiments within the scope of this technology.

### DESCRIPTION OF EXAMPLE EMBODIMENTS

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. In particular, the following description sets forth example embodiments and otherwise provides information that enables a person skilled in the art to make and use the subject matter set forth in the appended claims, but may omit certain details already well-known in the art. The following description is, therefore, to be taken as illustrative and not limiting. A non-limiting discussion of terms and phrases intended to aid understanding of the present technology is provided at the end of this Detailed Description.

The present technology provides wound dressings and compositions useful in wound dressing compositions. Preferably, the materials used in such dressings are physiologically acceptable, commensurate with a reasonable risk/benefit ratio when used in the manner of this technology according to sound medical practice.

### Carboxymethyl Cellulose

Wound dressing compositions of the present technology comprise a matrix which forms a gel when contacted with an aqueous medium, such as water, blood or wound exudate. The medium comprises a gel forming material such as a cellulose derivative containing carboxyl groups, such as a cellulose ether. Such derivatized cellulose materials may be made by processes known in the art, such as through an alkali-catalyzed reaction of cellulose with chloroacetic acid. In various embodiments, the matrix is operable to absorb 10 grams, 15 grams, 20 grams, or 25 grams of fluid (e.g., water, blood of wound exudate) per gram of material. In a preferred embodiment, the matrix can absorb 20 grams or less of fluid per gram of material.

Preferably, the matrix comprises carboxymethyl cellulose (“CMC”), wherein carboxymethyl groups are bonded to hydroxyl groups in the glucopyranose monomers that make up the cellulose. The CMC may be in salt form, comprising a physiologically acceptable cation, such as sodium (i.e., sodium carboxymethyl cellulose). CMC is commercially available, such as Walocel™ (sold by The Dow Chemical Company), Cekol® (sold by CP Kelco). In various embodiments, the matrix provides CMC fibers, as further discussed below.

CMC may be present in the composition at any level appropriate to result in the desired absorbency and rheological characteristics of the wound dressing composition. In general, the CMC may be present at a level of from about 50% to about 98% of the composition, or from about 60% to about 95%, or from about 70% to about 90%, of the wound dressing composition. (Unless otherwise indicated, all percentages herein are by weight of the wound dressing composition.)

### Oxidized Cellulose

The wound healing composition also comprises oxidized cellulose, preferably oxidized regenerated cellulose (ORC). Oxidized cellulose may be produced by the oxidation of cellulose, for example with dinitrogen tetroxide. This process converts primary alcohol groups on the saccharide residues to carboxylic acid group, forming uronic acid residues within the cellulose chain. The oxidation may not proceed with complete selectivity, and as a result hydroxyl groups on carbons 2 and 3 may be converted to the keto form. These ketone units introduce an alkali labile link, which at pH 7 or higher initiates the decomposition of the polymer via formation of a lactone and sugar ring cleavage. As a result, oxidized cellulose is biodegradable and bioabsorbable under physiological conditions.

The preferred oxidized cellulose for practical applications is oxidized regenerated cellulose (ORC) prepared by oxidation of a regenerated cellulose, such as rayon. ORC may be manufactured by the process described in U.S. Pat. No. 3,122,479, Smith, issued Feb. 24, 1964, incorporated herein by reference. ORC is available with varying degrees of oxidation and hence rates of degradation. The ORC may be used in the form of insoluble fibers, including woven, non-woven and knitted fabrics. In other embodiments, the ORC is in the form of water-soluble low molecular weight fragments obtained by alkali hydrolysis of ORC.

In certain embodiments, the oxidized cellulose is in the form of particles, such as fiber particles or powder particles, for example dispersed in a suitable solid or semisolid topical medicament vehicle. In some embodiments, the wound dressing compositions comprise ORC fibers, wherein a volume fraction of at least 80% of the fibers have lengths in the range of from about 20 μm to about 50 mm. In some embodiments, a volume fraction of at least 80% of the fibers have lengths in the range of from about 5 μm to about 1000 μm, or from about 250 μm to about 450 μm. In some embodiments, a volume fraction of at least 80% of the fibers have lengths in the range of from about 25 mm to about 50 mm. Desired size distributions distribution can be achieved, for example, by milling an ORC cloth, followed by sieving the milled powder to remove fibers outside the range. CMC fibers may be made by processes including those known in the art, such as by derivatization of cellulose fibers. CMC fibers are commercially available, for example, from Speciality Fibres and Materials Ltd.

### Silver

The wound dressing compositions comprise a safe and effective amount of silver. As referred to herein, a “safe and effective” amount of silver (or other material used herein) is an amount that is sufficient to have the desired effect (e.g., antimicrobial activity, with respect to silver), without undue adverse side effects (such as toxicity, irritation, or allergic response), commensurate with a reasonable benefit/risk ratio when used in the manner of this technology. The specific safe and effective amount of the silver may vary with such factors as the form of silver, the type and quantity of other materials in the composition, the intended use, and the physical condition of the subject on whom the wound dressings are used.

The silver may be present in the composition in metallic form, in ionic form (e.g., a silver salt), or both. Preferably, the silver is present in ionic form, such as in a complex with an anionic polysaccharide in the composition. In various embodiments, the wound dressing composition comprises a complex of silver and ORC (a “Silver/ORC Complex”). As referred to herein, such a complex is an intimate mixture at the molecular scale, preferably with ionic or covalent bonding between the silver and the ORC. The Silver/ORC Complex preferably comprises a salt formed between the ORC and Ag⁺, but it may also comprise silver clusters or colloidal silver metal, for example produced by exposure of the complex to light. Generally, the amount of silver in the Silver/ORC Complex may be from about 0.1% to about 50% by weight of the ORC, or from about 1% to about 40%, or about 2% to about 30% or from about 5% to about 25% by weight of the ORC. In various embodiments, the Silver/ORC Complex may be present in the wound dressing composition at a level of from about 0.5% to about 10%, or from about 0.5% to about 5%, or from about 1% to about 2%. For example, a dressing composition may comprise from about 1% to about 2% of a Silver/ORC Complex (by weight of the composition), wherein the Silver/ORC Complex comprises from about 200/% to about 300/% (e.g., about 25%) of silver by weight of the ORC.

The complex of an anionic polysaccharide and silver contained in the materials of the present invention can be made by treating the ORC with a solution of a silver salt. The ORC may be, for example, in the form of solid fibers, sheet, sponge or fabric. In certain embodiments, the anionic polysaccharide is a salt and the treatment therefore can be regarded as an ion exchange. In other embodiments, the anionic polysaccharide is at least partly in free acid form, in which case the silver salt is preferably a salt of a weak acid, for example silver acetate, whereby the anionic polysaccharide is at least partially neutralized by the silver salt. The reaction of ORC and silver can be carried out in water or alcohol alone but is preferably carried out in mixtures of water and alcohols. The use of a mixture of water and alcohol provides good solubility for the weak acid salts via the water, and the alcohol prevents the ORC from excessively swelling, distorting and weakening during the neutralization. Thus the physical properties of the material are retained. In various embodiments, the solution comprises water and alcohol at a ratio of from about 4:1 to about 1:4. Useful alcohols include methanol, ethanol, propanol, and isopropanol.

In various embodiments, the silver salt is the salt of silver with a weak acid. When using silver salts of weak acids, the silver ion is exchanged for a proton on the ORC and part of the salt is converted to weak acid. The mixture of acid and salt in the solution results in a buffered solution which maintains a fairly constant pH and controls the degree of reaction. An equilibrium reaction is established whereby the silver ions are bound to the acid portion of the ORC and also to the salt molecules. This partitioning of the silver ions prevents the neutralization of the ORC from going to completion. For example, using a stoichiometric amount of silver acetate may result in 65-75% degree of neutralization of the carboxylic acid groups on the ORC. This control of pH by creating a self-generating buffered solution and the use of methanol to control the swelling of the ORC, leads to a partially neutralized material in which the physical properties, e.g. tensile strength and shape of the ORC, are preserved. The amount of silver salt used is generally about equal to or up to twice the stoichiometric amount of carboxylic acid content of the ORC. Alternatively, a second charge of a stoichiometric amount of silver salt can be used if the reaction is recharged with fresh solvent and salt after the first charge reaches a constant pH. The material with elevated pH is then washed to remove the excess silver salt. Silver/ORC complexes useful herein, and methods of producing such complexes, are described in U.S. Pat. No. 8,461,410, Cullen et al., issued Jun. 11, 2013, incorporated by reference herein. Similar processes are described in U.S. Pat. No. 5,134,229, Saferstein et al., issued Jul. 28, 1992, incorporated by reference herein.

### Optional Components:

The wound dressing compositions may comprise one or more optional materials. Such optional components may include, for example, preservatives, stabilizing agents, plasticizers, matrix strengthening materials, dyestuffs, and actives.

In various embodiments, the wound dressing compositions contain a matrix strengthening material, which improves the handling characteristics of the CMC-containing matrix by, for example, decreasing its susceptibility to tearing. A preferred strengthening material comprises non-gelling cellulose fibers. Such “non-gelling” cellulose fibers are substantially water insoluble, produced from cellulose that has not been chemically modified to increase water solubility (as contrasted from carboxymethyl cellulose or other cellulose ethers). Non-gelling cellulose fibers are commercially available, such as Tencel® fibers (sold by Lenzing AG). Such fibers may be processed from a commercially-available continuous length, by cutting into lengths that are, in some embodiments, from about 0.5 to about 5 cm, or from about 2 to about 3 cm in length.

The non-gelling cellulose fibers may be present in the composition at any level appropriate to result in the desired physical characteristics of the wound dressing composition. In general, the non-gelling cellulose fibers may be present at a level of from about 5% to about 50% of the composition, or from about 10° % to about 40%, or from about 15% to about 25%, of the wound dressing composition. In some embodiments, the wound dressing compositions comprise the non-gelling cellulose fibers at a level such that the weight ratio of CMC:non-gelling cellulose fibers is from about 10:1 to about 1:1, such as about 8:1, 6:1, about 5:1, about 4:1, about 3:1, or about 2:1.

In various embodiments, the compositions are essentially free of water, wherein no water is added to the composition during its manufacture. However, wound dressing compositions may comprise up to 20/o water. Preferably, the compositions contain 10% or less, 8% or less, or 50% or water.

The wound dressing compositions may contain a dyestuff, which is preferably light-absorbing in the visible region 400-700 nm. Such dyestuffs may be operable to photochemically trap generated free radicals that could otherwise react with the silver in the present compositions, acting as photochemical desensitisers. In various embodiments, the antioxidant dyestuff is selected from the group consisting of aniline dyes, acridine dyes, thionine dyes, bis-naphthalene dyes, thiazine dyes, azo dyes, anthraquinone dyes, and mixtures thereof. For example, the antioxidant dyestuff may be selected from the group consisting of gentian violet, aniline blue, methylene blue, crystal-violet, acriflavine, 9-aminoacridine, acridine yellow, acridine orange, proflavin, quinacrine, brilliant green, trypan blue, trypan red, malachite green, azacrine, methyl violet, methyl orange, methyl yellow, ethyl violet, acid orange, acid yellow, acid blue, acid red, thioflavin, alphazurine, indigo blue, methylene green, and mixtures thereof. If present, the dyestuff may be present at a level of about 0.05% to about 5%, typically about 0.2% to about 2%.

The wound dressing composition may contain a plasticizer, such as glycerol or other polyhydric alcohol. If present, the plasticizer is present at a level of from about 0.5% to about 40%, or from about 5% to about 25%, or from about by weight, preferably 0-25% by weight.

The wound dressing composition may also comprise one or more additional active materials which aid in wound healing. Such actives include non-steroidal anti-inflammatory drugs (e.g. acetaminophen), steroids, antibiotics (e.g. penicillins or streptomycins), antiseptics other than silver (e.g. chlorhexidine), and growth factors (e.g. fibroblast growth factor or platelet derived growth factor). If present, such actives are typically present at a level of from about 0.1% to about 10%, or from about 1% to about 5%.

### Methods of Manufacturing

The wound dressing materials may be made by a variety of methods including methods known in the art. The present technology provides methods comprising admixing a Silver/ORC complex with carboxymethyl cellulose. In some methods the Silver/ORC complex is admixed with a mixture of carboxymethyl cellulose and non-gelling cellulose fibers.

In some embodiments, methods comprise providing a Silver/ORC complex, which is admixed with the carboxymethyl cellulose. Such “providing” may comprise obtaining a commercially-available Silver/ORC complex which is processed so as to produce silver-complexed ORC fibers that are then admixed with the CMC. Alternatively, or in addition, methods may comprise producing a Silver/ORC complex, such as by methods describe above.

Admixing the Silver/ORC Complex may be performed by any suitable method so as to, preferably, obtain a homogenous admixture of Silver/ORC Complex with CMC or a mixture of CMC with non-gelling cellulose fibers. In some embodiments, the Silver/ORC Complex comprises fibers having a length of from about 10 to about 70 mm, or from about 25 to about 50 mm, which are then needled into a mixture comprising CMC and non-gelling cellulose fibers.

In some embodiments, methods comprise forming a silver complex of ORC after the ORC has been mixed with CMC. Such methods generally include those discussed above for forming Silver/ORC Complexes. In some embodiments, such methods form complexes of silver with CMC, as well as with the ORC.

### Wound Dressings

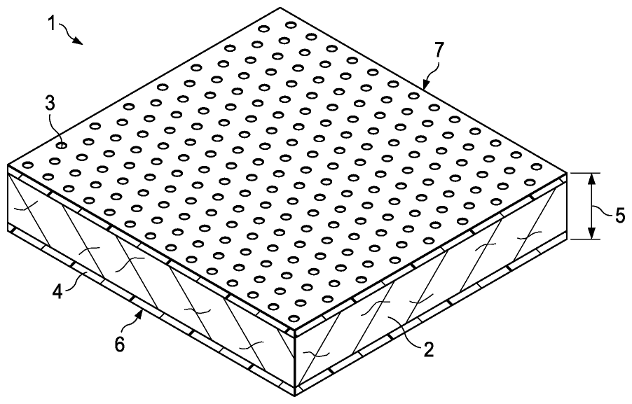

The present technology provides wound dressings comprising one or more wound dressing compositions as described above. In general, with reference to FIG. 1, such dressings 1 comprise an absorbent layer 2, wherein the absorbent layer comprises a wound dressing composition of the present technology. The absorbent layer 2 is preferably in substantially sheet form, i.e., having a generally planar structure with two opposite planar surfaces and a depth (or thickness) 5 orthogonal to the planar surfaces. For example, the wound dressing may have a wound facing surface 7 and an opposite back surface 6. The wound facing surface may have a surface area of from about 1 cm² to about 400 cm², from about 2 cm² to about 200 cm², or from about 4 cm² to about 100 cm². Such “planar” surfaces may have a variety of shapes, including square, rectangular, elliptical, circular or other geometries. It will be understood that the shape and area of the wound facing surface may be customized to the location and type of wound onto which the dressing is to be applied.

In various embodiments, the dressings comprise one or more additional layers 3, 4, also comprising sheet-form compositions. Such additional layers may perform any of a variety of functions in the dressings, including adherence of the absorbent layer to the wound or to surrounding tissues, increasing structural rigidity of the dressing, protection of the absorbent layer from contact with moisture or other materials in the environment in which the dressing is used, protection of a wound surface, eliminating or controlling transport of microbes from the wound (such as from the wound to the absorbent layer), and effecting delivery of actives or other materials to the wound surface. In various embodiments such additional layers are conformable to the wound surface and surrounding tissues, for example, being capable of bending such that the wound-facing surfaces of the dressing are in substantial contact with the wound and surrounding tissues.

In some embodiments, the wound dressing further comprises a backing sheet 4 having a wound-facing surface 6 and an opposite back surface. The backing sheet 4 may support the absorbent layer 2 on the wound-facing surface of the backing sheet, such that the back surface of the absorbent layer 2 is proximate to the wound-facing surface of the backing sheet 4. In some embodiments, the back surface of the absorbent layer 2 is in contact with, preferably adhered to, the wound-facing surface of the backing sheet 4.

Preferably, the backing sheet is substantially liquid-impermeable, although permeable to water vapor. Accordingly, in some embodiments, the backing sheet is not permeable to liquid water or wound exudate. Suitable backing sheets will preferably have a moisture vapor transmission rate (MVTR) of the backing sheet alone of 300 to 20,000 g/m²/24 hrs, preferably 500 to 10,000 g/m²/24 hrs at 37.5° C. at 100% to 10% relative humidity difference. Preferably, the backing sheet is also microorganism-impermeable.

Suitable polymers for forming the backing sheet include polyurethanes and poly alkoxyalkyl acrylates and methacrylates. In various embodiments, the backing sheet comprises a continuous layer of a high-density blocked polyurethane foam that is predominantly closed-cell. Backing sheet materials among those useful herein are disclosed in U.S. Pat. No. 3,645,835, Hodgson, issued Feb. 29, 1972, incorporated by reference herein. A suitable backing sheet material is the polyurethane film commercially available as Estane® 5714F (sold by The Lubrizol Corporation).

In various embodiments, the backing sheet thickness is in the range of from about 10 μm to about 1000 μm, or from about 20 μm to about 500 μm. The surfaces of the backing sheet may have a size and configuration such that an area of the backing sheet extends beyond the absorbent layer, i.e., wherein the backing sheet defines a marginal region extending from about 0.5 to about 60 mm, or from about 1 mm to about 50 mm, beyond one or more edges of the absorbent layer. The absorbent layer may be characterized as an “island” on the backing sheet. In various embodiments, the marginal region of the backing sheet (i.e., on the wound-facing surface of the backing sheet) is coated with an adhesive. Thus, when applied to a wound tissue, the marginal area may be used to adhere the dressing to tissues surrounding the wound.

Adhesives among those useful here include those known in the art, such as pressure sensitive adhesives. In various embodiments, the adhesive is a sensitive adhesive based on acrylate ester copolymers, polyvinyl ethyl ether, and polyurethane. Pressure sensitive adhesives among those useful herein are disclosed in U.S. Pat. No. 3,645,835, Hodgson, issued Feb. 29, 1972, incorporated by reference herein. The basis weight of the adhesive layer may be, for example, from about 20 g/m² to about 250 g/m², or from about 50 g/m² to about 150 g/m².

With further reference to FIG. 1, the wound dressings 1 may also comprise a top sheet 3 having a wound-facing surface 7 and a back surface, such that the wound-facing surface of the absorbent layer 2 is proximate to the back surface of the top sheet 3. The top sheet 3 is preferably permeable to wound fluids such as blood and wound exudate, allowing such fluids to be absorbed by the absorbent layer. In some embodiments (as generally exemplified in FIG. 1), the top sheet 3 is perforated, having a pore size that excludes passage of bacteria and other microbes.

The wound dressings are preferably sterile and packaged in a microorganism-impermeable container.

### Methods of Use

The present technology provides methods of treating a wound, comprising applying to the wound a wound dressing composition, preferably as a component of a wound dressing, as described above. The compositions and dressings may be used with any of a variety of wounds, such as those occurring from trauma, surgery or disease. For example, such wounds may be chronic wounds venous ulcers, decubitus ulcers or diabetic ulcers.

### Non-Limiting Discussion of Terminology

The headings (such as “Background” and “Brief Summary”) and sub-headings used herein are intended only for general organization of topics within the present disclosure, and are not intended to limit the disclosure of the technology or any aspect thereof. In particular, subject matter disclosed in the “Background” may include novel technology and may not constitute a recitation of prior art. Subject matter disclosed in the “Brief Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition or method.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Components may be also be combined or eliminated in various configurations for purposes of sale, manufacture, assembly, or use. Specific examples are provided for illustrative purposes of how to make and use the compositions and methods of this technology and, unless explicitly stated otherwise, are not intended to be a representation that given embodiments of this technology have, or have not, been made or tested. Equivalent changes, modifications and variations of some embodiments, materials, compositions and methods can be made within the scope of the present technology, with substantially similar results.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

As used herein, the words “preferred” or “preferable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

The example embodiments may also be described herein with reference to spatial relationships between various elements or to the spatial orientation of various elements depicted in the attached drawings. For example, such relationships or orientations as “top” or “bottom” assume a frame of reference consistent with an exemplary special orientation of a wound dressing. However, as would be recognized by those skilled in the art, this frame of reference is merely a descriptive expedient rather than a strict prescription as to the orientation of any given dressing as manufactured or used.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A wound dressing composition comprising:
(a) carboxymethyl cellulose, wherein the carboxymethyl cellulose is present in the dressing composition at a level of from about 50% to about 95%;
(b) non-gelling cellulose fibers, wherein the cellulose fibers are present in the dressing composition at a level of from about 5% to about 50%; and
(c) oxidized regenerated cellulose complexed with silver, wherein the complex of silver and oxidized regenerated cellulose complex is present in the dressing composition at a level of from about 0.5% to about 5%.

2. The wound dressing composition according to claim 1, wherein the silver is in an ionic form.

3. The wound dressing composition according to claim 1, wherein the complex of silver and oxidized regenerated cellulose comprises from about 10% to about 40% silver.

4. The wound dressing composition according to claim 3, wherein the complex comprises about 20% to 30% silver.

5. The wound dressing composition according to claim 1, wherein the complex is present in the dressing composition at a level of from about 1% to about 2%.

6. The wound dressing composition according to claim 1, wherein the carboxymethyl cellulose is present at a level of from about 70% to about 90%.

7. The wound dressing composition according to claim 1, wherein the cellulose fibers are present at a level of from about 15% to about 25%.

8. The wound dressing composition claim 1, wherein the composition further comprises a hydrogel.

9. The wound dressing according to claim 8, wherein the hydrogel is selected from the group consisting of polyurethane gels, hydroxyethyl cellulose, hydroxylpropyl cellulose, hydroxypropylmethyl cellulose, modified acrylamide polymers, alginates, pectin, galactomannans, chitosans, gelatins, hyaluronates, and a mixture of any two or more thereof.

10. The wound dressing composition according to claim 1, further comprising collagen.

11. The wound dressing composition according to claim 1, further comprising a wound healing active material.

12. The wound dressing composition according to claim 11, wherein the wound healing active material is an antimicrobial.

13. A wound dressing comprising an absorbent layer comprising a wound dressing composition according to claim 1.

14. The wound dressing according to claim 13, wherein the absorbent layer is in sheet form having a wound facing surface and an opposite back surface, and wherein the wound facing surface has a surface area of from about 1 cm² to about 400 cm².

15. The wound dressing according to claim 14, further comprising a backing sheet having a wound-facing surface, wherein the wound-facing surface of the backing sheet substantially covers the back surface of the absorbent layer.

16. The wound dressing according to claim 15, wherein the backing sheet has a surface area larger than the surface area of the back surface of the absorbent layer, having a marginal region extending from about 1 mm to 50 mm beyond two or more edges of the absorbent layer.

17. The wound dressing according to claim 16, wherein the wound-facing surface of the backing sheet is coated with an adhesive in the marginal region.

18. The wound dressing according to claim 14, further comprising an apertured top sheet substantially covering the wound facing surface of the absorbent layer.
